(19) United States
(12) Patent Application Publication
(10) Pub. No.: US 2008/0131947 A1
(43) Pub. Date: Jun. 5, 2008

(54) Treatment of Cellulosic Material for Ethanol Production

(75) Inventor: Jeffrey Bruce Wicking, Bayview (AU)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: Cellencor, Inc., Ames, IA (US)

(21) Appl. No.: 11/948,678

(22) Filed: Nov. 30, 2007

Related U.S. Application Data
(60) Provisional application No. 60/868,115, filed on Dec. 1, 2006.

(51) Int. Cl. C12N 13/00; C12P 7/10

(52) U.S. Cl. 435/165; 435/173.2

(57) ABSTRACT

The invention is directed to a process for converting cellulosic material to ethanol comprising adding an enzyme capable of releasing sugars to cellulosic material to form a cellulosic material and enzyme mixture; treating the mixture with microwave energy to enhance enzymatic digestion of the cellulosic material by the enzyme to release sugars; and carrying out a fermentation reaction on the treated mixture to form ethanol.

### RELATED APPLICATIONS

This application claims the benefit under 35 U.S.C. §119(e) of provisional U.S. Patent Application Ser. No. 60/868,115, filed Dec. 1, 2006, the entire contents of which are incorporated herein by reference.

### FIELD

The present invention relates to treatment of cellulosic material by microwave energy to improve breakdown and enzyme digestion of the treated material for ethanol production.

### BACKGROUND

Plant material wastes (biomass) are made up of five main components: cellulose, hemicellulose, lignin, crude protein and ash. Cellulose is generally a linear, unbranched glucose-based homopolymer, i.e., a polysaccharide, of relatively high molecular weight. Hemicellulose is typically a branched and/or unbranched polymer of D-glucose, D-mannose, L-arabinose and D-xylose of about 100-200 sugar residues per polymer chain. Lignins are amorphous crosslinked phenolic polymers that occur uniquely in vascular plants and comprise 20-30% of most wood.

Processing of biomass is important in several industries such as fuel and ethanol production, waste management, pulp and paper, food manufacture, and energy production among others. For example, it is known to hydrolyze cellulosic materials into monosaccharides for varying purposes including feed stocks for other chemicals, food stuffs, fuels, and the like. In addition, conversion of biomass to sugars usable directly as food or as chemical reagents is an interest in planning long-term space missions. In many agricultural products, only half of the crop is edible. Of the inedible portion, approximately 50-68 percent is polysaccharide which can be reduced into fermentable sugars. The remainder is primarily unusable lignin.

Reduction of polysaccharides by hydrolysis is well known in the art. Two basic methods are generally used: (1) chemical treatment, e.g., reduction using an acid catalyst; and (2) biological breakdown using enzymes or microorganisms. Such methods generally include one or more pre-treatments to increase hydrolysis reaction rate and yield. Pre-treatments typically increase the availability and surface area of reducible polysaccharides by disturbing the physical and molecular structure of the feed material and/or fractionating the lignocellulosic material into its lignin, hemicellulose and cellulose components.

In the United States of America (US), ethanol produced from grains is commonly used as an automotive fuel additive. The co-products of the ethanol production process are commonly used as animal feed. While the worldwide demand for ethanol used in automotive fuel is rapidly increasing, this is not the case for the co-products of ethanol. The adoption of techniques to maximize the value of these co-products while minimizing their production cost is critical to the continued success of the ethanol industry.

Large scale US production of ethanol began in the late 1970's, and except for a brief drop in the mid-1990's, production has grown steadily. Since 2001, the rate of growth has accelerated rapidly, such that in 2003, production reached 2.81 billion gallons. Several factors have influenced the current increasing demand for ethanol. These include high petroleum prices as well as the need for environmentally friendly fuel oxygenates to replace Methyl Tertiary Butyl Ether (MTBE).

U.S. Pat. No. 5,196,069 is directed to an apparatus and method to convert cellulosic waste material in the presence of an organic acid into sugars under superatmospheric pressure. This invention was particularly suitable for processing being carried out in outer space. This invention, however, is not particularly suitable for industrial applications such as ethanol production from cellulosic material.

With the increase in the desire to produce ethanol from cellulosic material, there is a need to improve yields and enhance the enzymatic processes involved.

The present inventor has found a pre-treatment with microwave energy can provide improved yields of ethanol production without the need to carry out the process under superatmospheric pressure.

### SUMMARY

A first embodiment provides a process for enhancing release of sugars in alcohol fermentation comprising adding an enzyme to the cellulosic material and treating the cellulosic material and enzyme mixture with microwave energy to enhance enzymatic digestion of the cellulosic material.

A second embodiment provides a process for converting cellulosic material to ethanol comprising: (a) adding an enzyme capable of releasing sugars to cellulosic material to form a cellulosic material and enzyme mixture; (b) treating the mixture with microwave energy to enhance enzymatic digestion of the cellulosic material by the enzyme to release sugars; and (c) carrying out a fermentation reaction on the treated mixture to form ethanol.

A third embodiment provides a process for enhancing release of sugars from cellulosic material for fermentation comprising: (a) adding to cellulosic material an enzyme capable of releasing sugars from cellulosic material to form a reaction mixture; (b) treating the reaction mixture with microwave energy for sufficient time to enhance release of fermentable sugars from the cellulosic material; and (c) carrying out a fermentation reaction on the treated reaction material using yeast to form alcohol.

Preferably, the cellulosic material is selected from any suitable cellulosic biomass feedstock including agricultural wastes (such as corn stover, cereal straws and sugarcane bagasse), plant waste from industrial processes (such as saw dust, paper pulp) and energy crops especially grown for fuel production such as switch grass, components thereof, and mixtures thereof. More preferably, the cellulosic material is corn stover.

The enzyme is preferably selected from amylase, alpha amylase, glucoamylase, phytase, phosphatase, carbohydrate hydrolyzing enzymes, xylanase, cellulase, hemi-cellulase, and mixtures or combinations thereof. Preferably, the enzyme is an amylase, alpha-amylase or glucoamylase. However, the skilled artisan will realize that the claimed subject matter is not limited to the enzymes explicitly listed herein, but may encompass other enzymes of use to break down plant biomass into simpler components, so long as the activity of the enzyme on the substrate is enhanced by microwave treatment.

The enzyme can be added at a range of about 100 g to 10000 g per metric tonne (1000 kg), typically about 500 g to 5000 g per metric tonne. Preferably, the enzyme is added at about 1000 g to 2000 g per 1000 kg cellulosic material. It will be appreciated that the amount and type of enzyme will depend on the cellulosic material being treated or processed.

Preferably, the microwave energy has a frequency in the order of 2.45 GHz or in the 900 MHz frequency range. It will be appreciated that the frequency can vary, depending on the approved microwave frequencies used in different countries or regions of the world. The actual frequency used does not have a specific material affect on the claimed methods.

Preferably, treating with microwave energy is carried out such that the temperature of the enzyme mixture is effectively controlled. Typically, the temperature is from about 50° C. to less than about 100° C. Preferably, the temperature is about 60° C. to 90° C., or more preferably about 65° C. to 78° C. For many amylases, the temperature is preferably about 70° C.

Many enzymes have a preferred temperature range for activity and the temperature can be selected for a particular enzyme or enzyme mixture. As some suitable enzymes may be heat intolerant or temperature sensitive, it is desirable not to inactivate the enzymes with too high a temperature. It will be appreciated that determining the desired or controlled temperature is within the skill of the operator.

Treating with microwave energy can be carried out in a continuous or batch manner.

Preferably, treating with microwave energy is carried out for up to about 10 minutes per kg cellulosic material.

The claimed methods utilise microwave energy or irradiation to enhance the action of an enzyme upon its substrate. The microwave frequency used in illustrative embodiments is in the order of 2.45 Ghz. This frequency is the one available for use in Australia but other frequencies such as 915 MHz may also be used in the claimed methods. The amount of microwave energy required can be dependent upon the water moisture present within the cellulosic material and enzyme mixture. The amount of microwave energy used is also dependent upon the type of material being treated as different cellulosic material can have different dielectric constants. Material with high dielectric constants absorb microwave energy preferentially and are therefore heated or acted upon before compounds with lower dielectric constants. However, other heating mechanisms may be used to bring the enzyme solution and substrate up to the activation temperature of the enzyme at which point the microwave treatment can then be applied.

In a preferred form, the microwave energy is applied such that the temperature of the reaction mixture is effectively controlled. Furthermore, it has been found that it is preferable to apply the microwave energy to the mixture in a continuous manner.

Time of treatment will vary depending on the enzyme, substrate and the volume of material to be treated. About 10 minutes of microwave treatment has been found to be particularly suitable for rice bran treatment with water and enzymes added.

The fermentation reaction is typically carried out by microbial fermentation. Preferably, the microbial fermentation utilizes yeasts.

The claimed process can be adapted for current methods of ethanol production such as wet milling and dry milling.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the term “about” is understood to mean plus or minus 10 percent of a stated numeric value. For example, “about 100” would cover all numbers between 90 and 110.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this specification.

In order that the present invention may be more clearly understood, preferred embodiments will be described with reference to the following drawings and examples.

### BRIEF DESCRIPTION OF THE DRAWINGS

FIG. 1 shows a schematic of a Wet Milling Process for ethanol production from grain.

FIG. 2 shows a schematic of a Dry Milling Process for ethanol production from grain.

### DETAILED DESCRIPTION

Ethanol has become an important renewable energy source. In 2006 over 40% of the gasoline consumed in the United States of America (US) was a blend containing at least 10% ethanol content. Almost all ethanol is produced by the fermentation and distillation of biomass, particularly grains. In the US, corn is currently the most widely used feedstock.

Three important factors in renewable energy production are a) minimizing energy use in order to maximize the net energy gain, b) minimizing negative environmental effects incident to the production process, and c) maximizing the value of co-products.

### Fuel Ethanol Production

There are two main industrial methods of producing fuel ethanol, wet milling and dry milling. The overwhelming majority of ethanol plants in the US use the dry milling process.

In wet milling, the incoming corn is first inspected and cleaned. Then it is steeped in water for 30 to 40 hours to begin breaking the starch and protein bonds. The next step is a coarse grind to separate the germ from the rest of the kernel. The remaining slurry consisting of fiber, starch and protein is finely ground and screened to separate the fiber from the starch and protein. The starch is separated from the remaining slurry in hydrocyclones. The starch is then used for the fermentation process. The other co-products are typically dried before use. Wet milling is a capital intensive and complex process used primarily in a few very large industrial processing plants.

In dry milling, the entire corn kernel is milled into a “meal”, and processed without separating out the various component parts of the grain. The meal is slurried with water to form a “mash.” A heat stable enzyme (typically α-amylase) is added to the mash to convert the starch to dextrose. In the next step, “liquefaction”, jet cookers inject steam to cook the mash above 100° C. This reduces bacteria levels and breaks down the starch granules in the kernel endosperm. The slurry is allowed to cool to about 80° C. and more α-amylase enzyme is added to further fragment the starch polymers. Finally, in a process called “saccharification”, the slurry is cooled to about 30° C. and a different enzyme (typically glucoamylase) is added which begins the conversion of the starch to sugar (glucose) which continues through the microbial fermentation process.

Both methods use similar fermentation processes. The starch or slurry is put in a fermentation tank, and yeast is added to convert the simple sugars to ethanol. After fermentation, the liquid slurry has an ethanol content of about 10% to 12% by weight. The slurry is distilled which produces a product which is about 95% ethanol by weight. The remaining water is typically removed using molecular sieves.

The residual product after distillation, referred to as stillage, consists of liquids (mostly water and some ethanol) and corn solids. A centrifuge is used to separate much of the liquid (called thin stillage) from the solids (called wet cake).

Some of the thin stillage is recycled to the beginning of the process. The remainder is processed by an evaporator to produce a thickened co-product called syrup. Most often, the syrup is blended back into the wet cake. After drying, the product is thus referred to as “distillers' dried grain with solubles”, or DDGS. Some local demand as animal feed may exist, most of the DDGS must be dried to 12% or less moisture content because otherwise the wet cake has a storage life of only two or three days. A large amount of DDGS is produced; a typical 50 million gallon per year dry milling ethanol plant will produce 166,000 dry tons of DDGS per year. The value of the DDGS can be critical to the economic success of the plant.

### Current Methods of Ethanol Production

The properties and value of ethanol co-products are affected by the techniques employed to accomplish starch extraction during the production process. There are two major methods of ethanol production common today. These are typically referred to as “Wet Milling” and “Dry Milling,” each of which is briefly described below.

### Wet Milling and its Co-Products

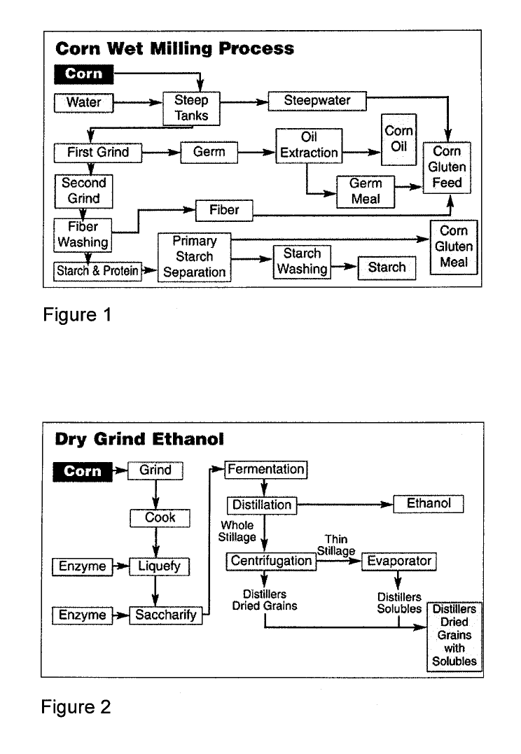

The Wet Milling method separates corn into its four major components: fiber, protein, oil and purified corn starch. The process isolates steep water, fiber, germ meal and gluten, all of which can be used in animal feed products. The flowchart shown below presents the Wet Milling process in detail (see FIG. 1).

Although Wet Milling has higher capital and energy costs than does Dry Milling, these costs are offset by the production of a wider range of products. Wet Milling is typically used by larger, more established companies.

### Wet Milling Co-Products

The four co-products listed here represent about twenty-five to thirty percent of the corn that is processed.

Corn gluten feed is an intermediate protein product that is rich in highly digestible fiber. It may or may not contain the condensed corn extractives. This product is sold either wet or dry. The dry form combines bran and condensed extractives (sometimes germ meal). The dried corn gluten feed is then made into pellets to facilitate handling. Corn gluten feed analyzes typically as 21% protein, 2.5% fat and 8% fiber. Wet corn gluten feed (45% dry matter) is similarly combined but not dried. It is a perishable product in six to ten days, and must be fed or stored in an anaerobic environment. In both forms (wet and dry), this co-product is widely used as a complete feed for dairy and beef cattle, poultry, swine and pets.

Corn gluten meal is a high-protein concentrate typically supplied at 60% protein, 2.5% fat and 1% fiber. It is a valuable source of methionine. Corn gluten meal also has a level of xanthophylls, which offers an efficient yellow pigmenting ingredient for the manufacture of poultry feed. Corn gluten meal also is excellent cattle feed, providing a high level of rumen bypass protein.

Condensed corn fermented extractive, or corn steep liquor, is a high-energy liquid feed ingredient. The protein value analyzes at 25% on a 50% solids basis. This product is sometimes combined with the corn gluten feed. It may also be sold as a pellet binder and is a source of B-vitamins and minerals.

Corn germ meal is golden-yellow and is mainly gluten, the high-protein portion of the corn kernel. Corn gluten meal typically analyzes at 20% protein, 2% fat and 9.5% fiber. It has an amino acid balance that makes it valuable in poultry and swine rations. It is also used as a carrier of liquid feed nutrients.

### Enzyme Digestion

Since the 1950's, enzymes have played an increasing role in processing corn starch. Enzymes now perform the same reaction under relatively mild temperatures and pressures that previously required the use of acid combined with high temperatures and pressures. Breaking down starch molecules with enzymes is a two stage process. Initially, alpha-amylase splits the large amylose and amylopectin molecules that make up the starch into soluble dextrin fragments. The resulting starch slurry has a gravy-like consistency.

The enzyme glucoamylase saccharifies the gravy-like dextrins, hydrolyzing the polymers into their individual dextrose units. Successive units are cleaved off the ends of dextrins, including those at amylopectin branch points. The dextrose produced may be processed into finished syrup, dry dextrose, or may be fermented to produce ethanol for fuel or beverage use.

### Dry Milling and its Co-Products

Dry Milling refers to the Dry Grind Ethanol Process that is used by most farmer-owned ethanol production facilities (see FIG. 2). This process employs a minimal amount of grinding to expose the starch that is consumed by ethanol production.

The two primary co-products from this process are “distillers solubles” and “distillers grains”. The thirty percent of the corn kernel made up of protein, fat, minerals, and vitamins are not consumed in ethanol production. Dry Milling concentrates these components into distillers' grains, which are commonly used for animal feed.

As in Wet Milling, alpha-amylase and glucoamylase are used to break down the starch into simple sugars for fermentation. Corn typically has low levels of soluble nitrogen, slowing yeast growth and requiring a longer fermentation period or the addition of urea. Adding a small amount of protease enzyme also helps yeast to access nitrogen and reduces the fermentation time, minimizing the need for urea.

Natural enzymes are also active during the fermentation process. Yeast produces phytase, an enzyme that converts the phytic acid found in plant material into a form of phosphorus that can be used by the yeast. Phytase is so effective at converting phosphorous into a form that can be used by animals that it is increasingly used as an additive for poultry and swine feed containing dried distillers grain with solubles (DDGS). However, phytase is sensitive to temperatures above 100° C. Naturally occurring phytase remaining in the distillers grains is denatured and inactivated by current drying techniques.

As FIG. 2 shows, the ethanol distillation produces whole stillage, a mixture of suspended grain solids and water. Whole stillage has been described by others as having the rough consistency of thick chicken noodle soup which has gone through a blender. Due to its high nutrient levels, whole stillage is prone to rapid spoilage and requires immediate processing in order to minimize losses.

Passing whole stillage through a centrifuge allows for the rapid separation of a large quantity of water from the distillers grains. The solids generated by this process are commonly referred to as “wet cake.”

The fluid discharged from the centrifuge is referred to as “thin stillage.” This discharge includes all the water removed from the wet distillers grains and the water-soluble portions of the distillers' grain. The fraction of distillers' grain present in thin stillage is extremely stable in solution. This characteristic makes it possible to remove a large percentage of water from this mixture without an excessive amount of material building up in the evaporator.

Multiple effect evaporators reuse the steam condensate from previous stages for additional heating effect, by operating at progressively lower pressures. The use of a three-effect evaporator allows one pound of steam (equivalent to roughly 1,000 BTU) to remove several pounds of water.

The final stage of drying wet cake utilizing the current drying technology is the most difficult and energy intensive step, requiring 1,220 to 1,350 BTU to remove one pound of water. Developing more efficient drying techniques that preserve and/or enhance the nutritional values of DDG and DDGS is critical to expanding the market for ethanol co-products.

The Dry Mill process is typically preferred by smaller startup companies due to its lower first cost and energy consumption.

### Microbial Fermentation

Fermentation of treated cellulosic material is carried out using microorganisms such as yeasts in the usual manner known to the art of ethanol production.

### Industrial Microwave Ovens

Industrial microwave ovens have been widely used for at least fifty years. Principle applications include cooking and processing food for human consumption, and drying various materials such as wood products. A person skilled in the art would appreciate how to the select, build or adapt a suitable microwave oven for the present invention.

One type of industrial oven is a seamless welded steel box approximately one square meter, however, dimensions can vary. The front of the oven has an access door and the oven is designed to prevent leakage of microwave energy.

For batch treatment, material is placed in the oven and treated with microwave energy for an appropriate time.

At the top of the oven is one or more rotating dipole antennas which emit microwave power into the oven cavity. The rotation of the antenna is to ensure even distribution of microwave energy throughout the oven. The antenna is connected via rectangular waveguide to a transmitter unit which generates the microwave energy. The oven may be fed by one or two transmitters, depending on the design capacity.

The transmitter generates microwave energy using a water cooled magnetron tube. Each transmitter typically generates up to 75 kilowatts of power with a conversion efficiency of about 85%.

The high voltage power supply for the magnetron steps up 480 volt 3 phase mains voltage (via a power control circuit) to 10 kilovolts, which is converted to DC current using a high voltage rectifier bridge. In this application a frequency of 915 megahertz is used, which allows deep material penetration and high power generation.

The output of the magnetron tube is connected via a three port device called a “circulator”. It routes radio frequency (RF) energy to the oven feed waveguide and/or a water cooled dummy load. The circulator provides protection to the system by automatically diverting reflected (reverse) RF energy to the dummy load. This could occur due to an insufficient load in the oven, arcing in the oven, damage to the waveguides or oven, or other fault conditions.

The transmitter cabinet can also house a process control computer and associated electrical controls. The computer communicates with a touch screen LCD user interface located on the oven. The computer automates the operational, monitoring, and safety features of the transmitter and the associated oven. The computer can precisely control the microwave power output with one kilowatt resolution in either pulsed or continuous modes.

Another type of industrial oven system particularly suitable for the present invention comprises several separate cooking ovens (cavities) as described above arranged in a horizontal feed line. In this arrangement, typically two, three, four, five or more ovens are used on each line, depending on the design capacity. Each oven is a seamless welded steel box approximately one square meter, however, dimensions can vary. Each oven has entry and exit doors or ports and the ovens are designed to prevent leakage of microwave energy.

For continuous treatment, a conveyor belt or the like is used to move material and extends through ports located on both sides of the each oven. The oven boxes can be connected by enclosed plenums which the belt moves through. The first and last ovens have pin-type radio frequency chokes which prevent the leakage of microwave energy so the ends of the belt may be in the open for product loading and unloading. One or more screened vent openings can be provided for removal of exhaust vapors. These vents are connected to high capacity blowers and a duct system.

In continuous treatment, material to be treated is passed through one or more ovens and that passage time allows the appropriate microwave treatment to be achieved.

### Microwave Treatment

In conventional heating, the heat source causes the molecules to react from the surface toward the center, so that successive layers of molecules are heated in turn. This process results in every molecule in the material being heated to some degree and commonly results in the outer layer of the material becoming over-dried.

In an effort to prevent damage to nutrients from over-drying, rotary kiln and ring dryers attempt to expose all surfaces of the granular material being dried to the heated air stream. This maximizes both heat transfer into the particle and mass transfer of water out of the particle.

Microwaves utilize alternating electromagnetic waves that move at the speed of light to transmit energy. Unlike most molecules, the unbalanced electrical charge on polar molecules makes them react to electromagnetic fields.

As microwaves pass through a material, polar molecules move to align their positive and negative charges with the electric field. Switching the field at 915,000 times per second forces polar molecules such as water, sugar and fat to oscillate. The molecular motion produces a heating effect due to friction, between vibrating molecules and the surrounding material. Due to the speed at which the microwaves travel, the heating effect is uniform throughout the volume of homogeneous materials.

Among all types of heating, dielectric (microwave) heating is the only one that can produce a higher temperature inside a product than on its surface. The peak temperature at the surface will never exceed the temperature required to allow for water to evaporate from its surface.

### Industrial Microwave Construction

Industrial microwave ovens are typically constructed of steel or stainless steel. The metallic construction of the oven traps the microwaves and reflects it back into the product being processed.

The use of a variable speed conveyor belt and computerized control are used to give microwave systems excellent control of the drying process. By adjusting the belt speed, the moisture content in the final product can be more tightly, controlled than with other types of heaters or dryers.

Microwaves are limited in the thickness of material that they can effectively heat, to roughly three inches in depth. Aside from this constraint the configuration of the microwave oven is extremely flexible. The width of material can range from four feet wide with a single antenna to fifteen feet wide with multiple antennas located over portions of the conveyor belt.

The use of a rotating antenna installed at the top of the oven cavity helps produce a uniform heating effect.

Rectangular aluminum tubes are used to conduct the microwaves from the transmitter to the antenna in the dryer. These connectors are commonly referred to as “wave guides” and can be bolted together using standard elbows and other fittings.

The microwave transmitter generates the microwave energy of a given frequency and a maximum power output that is launched into the antenna. Transmitter components include a magnetron, electromagnet, power supply, circulators, water load, and controls.

The control system monitors and controls the performance of the system and facilitates programming/automation as well as in-process adjustments of variables such as power output and process speed. It also monitors the operation of the system with regard to safe operating parameters and can shut the system down in the unlikely event that unsafe conditions are detected.

Microwave apparatus can include a “choking” mechanism at the entry and exit to the conveyor system. This feature reduces microwave leakage to below detectable levels. The microwave doors and seals are the components that are most prone to damage. Old or faulty door seals are the most common causes of microwave leakage. Mechanical abuse, a build-up of dirt, or simple wear and tear of continued use can cause door seals to be less effective.

Safety tips for installation and maintenance of microwave dryers include:

Take special care to ensure that no damage occurs to the part of the oven making contact with the door or door seals.

Ensure that the microwave is disconnected from electrical power before reaching into any accessible openings or attempting any repairs.

Ensure that the adjustment of applied voltages, replacement of the microwave power generating component, dismantling of the oven components, and refitting of waveguides are undertaken only by persons who have been specially trained for such tasks.

Do not by-pass the door interlocks.

Do not test a microwave power-generating component without an appropriate load connected to its output. The power generated must never be allowed to radiate freely into occupied areas.

### Methods

### Microwave Technology

Microwave treatment consists in the transformation of the alternating electromagnetic field energy to the thermal energy by affecting polar molecules of a material. Compared to conventional heating, microwave treatment has substantial benefits including speed, temperature homogeneity and high levels of energy absorption into the heated material.

### Enzymes

Enzymes suitable for use in the present invention include amylases, alpha amylase, glucoamylases, phytases, phosphatases, carbohydrate hydrolyzing enzymes, xylanases, cellulases, hemi-cellulases, and mixtures or combinations thereof.

### Soluble and Insoluble Non-Starch Polysaccharides (NSP) Analysis

The levels of soluble and insoluble NSP of the rice bran were determined using a stream-lined assay. Samples (100 mg) were ground to pass a 0.5 mm screen. Samples were then extracted twice with an ethanol: water mixture (85:15; 2 ml) at 80° C. for 5 minutes to remove soluble sugars.

### Hydrolysis

The residue was hydrolysed for 2 hours at 100° C. using 3 ml of 1M H₂SO₄. A 0.4 ml aliquot of hydrolysate was transferred to a 30 ml culture tube to which 0.10 ml of 28% NH₃ was added. The 50 μl of Inositol (4 mg/ml) and 50 μl of allose (4 mg/ml) were added as internal standards. The mixture was dried under nitrogen at 40° C.

### Reduction

The monosaccharides were reduced using sodium borohydride as follows. To the mixture of sugar hydrolysate and internal standards, water (0.2 ml), absolute ethanol (0.2 ml), and 3M ammonia (1 drop) were added. After thoroughly mixing, freshly prepared NaBH₄ (prepared by dissolving 50 mg sodium per ml 3M NH₄OH) (0.3 ml) was added. The tubes were then capped and incubated in a water bath at 40° C. for 1 hour.

### Acetylation

To the reduced mixture, 5-7 drops of glacial acetic acid were added to decompose the excess of NaBH4. Then 0.5 ml 1-methylimidazole and 5 ml acetic anhydride were added and mixed, and left for 10 minutes at room temperature. Absolute ethanol (0.8 ml) was added, mixed and left for 10 minutes at room temperature to effect acetylation of the sugars. The samples were then placed in an ice bath, and to each tube 5 ml H₂O was added to decompose any excess of acetic anhydride. Five ml of 7.5M KOH were added, the tubes were capped and gently mixed six times by inversion. Another 5 ml of 7.5M KOH were added, capped and mixed again. At this stage a clear ethyl acetate top layer was visible; this layer was then transferred into a 4 ml vial by using a Pasteur pipette and was evaporated to dryness under N₂. The evaporate was then re-dissolved in 0.4 ml ethyl acetate and the sugars were quantified with a Varian 3400CX gas chromatographic instrument.

Duplicate samples were hydrolysed and the derivable products were determined twice. The levels of polymeric arabinoxylans were assumed to be comprised of a xylan backbone with arabinose side chains. The levels of arabinoxylans were calculated from the levels of the component sugars using a polymerisation factor of 0.88 to account for the water condensation. Other polysaccharides (glucan, galactan and mannan) were assumed to be linear polymers and levels were calculated using a polymerisation factor of 0.9.

### Free Sugar Analysis

Samples (100 mg) were extracted twice with diethyl ether (5 ml) to remove fat and pigments and were centrifuged at 3000 g for 15 min. The supernatants were discarded. The residues were then extracted with 80% ethanol and centrifuged (3000 g; 15 min). The supernatants were taken and dried under N2 and hydrolysed in 1 M H₂SO₄ at 100° C. for 2 h. Reduction and acetylation were the same as that described for soluble and insoluble NSP determination.

### EXPERIMENTS

### Experiment 1

Aim: To assess the impact of sugar release when varying the volume of enzyme supplement and the time of microwave treatment.

Method: Twelve lots of 5 kg rice bran samples with the enzymes (Biofeed Plus, Novo Nordisk, a carbohydrase preparation produced by submerged fermentation of Humicola insolens. The enzyme hydrolyzes arabino-xylans and beta-glucans into oligosaccharides and some mono-, di- and trisaccharides. Biofeed Plus also contains other carbohydrase activities including cellobiase, hemi-cellulase and cellulase) added to eight of the samples at two different levels (recommended dosage rate of 500 g/tonne and 1000 g/tonne). Five litres of water were added to each sample prior to microwave treatment. All samples were taken to 65° C. and then held at temperature for either 5 (all samples with prefix—MV) or 10 minutes (all samples with prefix—MMV), after which the temperature was increased to 85° C. and held for a further five minutes to de-activate the enzyme. The microwave frequency used was 2.45 GHz. Results are shown in Table 1.

The rice bran used in this experiment contained 25% NSP that were predominantly insoluble. Approximately 50% of the NSP were cellulose and the rest arabinoxylans.

TABLE 1
Effects of enzyme supplementation and microwave treatment
on the carbohydrate composition of rice bran

| | | | | | | | Total |
|---|---|---|---|---|---|---|---|
| | | | | | | | Sugars |
| | | | | | | | Released |
| SAMPLE | RIB | ARA | XYL | MAN | GAL | GLU | (g/kg) |
| Control 1 | 0 | 0 | 0 | 2.72 | 0 | 5.44 | 8.15 |
| Control 2 | 0 | 0 | 0 | 1.80 | 0 | 4.44 | 6.23 |
| Mean | 0 | 0 | 0 | 2.26 | 0 | 4.92 | 7.19 |
| BF500-MV-1 | 0.41 | 0.71 | 0.51 | 3.30 | 1.80 | 24.80 | 28.49 |
| BF500-MV-2 | 0.35 | 0.66 | 0.44 | 2.60 | 1.56 | 24.79 | 27.34 |
| Mean | 0.38 | 0.69 | 0.48 | 2.95 | 1.68 | 24.79 | 27.91 |
| BF500-MMV-1 | 0.53 | 0.25 | 0.33 | 5.67 | 1.49 | 38.80 | 42.34 |
| BF500-MMV-2 | 0.49 | 0.34 | 0.22 | 4.82 | 1.07 | 34.36 | 37.15 |
| Mean | 0.51 | 0.29 | 0.27 | 5.25 | 1.28 | 36.58 | 39.75 |
| BF1000-MV-1 | 0.32 | 0.89 | 0.74 | 2.48 | 1.69 | 20.00 | 23.47 |
| BF1000-MV-2 | 0.39 | 0.42 | 0.25 | 3.16 | 1.42 | 23.19 | 26.02 |
| Mean | 0.35 | 0.65 | 0.49 | 2.82 | 1.55 | 21.59 | 24.74 |
| BF1000-MMV-1 | 0.48 | 0.55 | 0.49 | 3.84 | 1.72 | 30.55 | 33.83 |
| BF1000-MMV-2 | 0.50 | 0.35 | 0.21 | 4.73 | 1.28 | 34.19 | 37.13 |
| Mean | 0.49 | 0.45 | 0.35 | 4.28 | 1.50 | 32.37 | 35.48 |
| CRLT-MV1 | 0.39 | 0.58 | 0.38 | 3.37 | 1.55 | 24.85 | 27.98 |
| CRLT-MV2 | 0.32 | 0.45 | 0.32 | 3.20 | 1.24 | 23.04 | 25.69 |
| Mean | 0.35 | 0.51 | 0.35 | 3.29 | 1.39 | 23.95 | 26.84 |
| CRLT-MMV-1 | 0.67 | 0.32 | 0.25 | 5.78 | 1.25 | 41.40 | 44.55 |
| CRLT-MMV-2 | 0.57 | 0.37 | 0.28 | 4.89 | 1.34 | 36.32 | 39.36 |
| Mean | 0.62 | 0.34 | 0.27 | 5.33 | 1.29 | 38.86 | 41.95 |

Results: The microwave treatment released mainly arabinose (ara), xylose (xyl) and glucose with trace amounts of ribose (rib), galactose (gal) and mannose (man). It appears the beneficial effect came mainly from the microwave treatment per se rather than the enzyme, indicating a possible de-activation of the enzyme during the microwave treatment. The interesting result from this trial was that a longer period (10 minutes) of microwave treatment had significantly higher amounts of sugar release (32.26% more) compared to the samples that had undergone a shorter (5 minutes) treatment. It can also be concluded that the amount of sugars released is independent of enzyme concentration. In this experiment the sample size was increased from the 200 g samples from earlier experiments to 5 kg samples, yet the increased sugar release effect was similar.

### Experiment 2

Aim: To determine whether the volume of feed processed and the time of soaking alters the amount of sugars released from the rice bran.

Method: Four lots of 20 kg rice bran samples with enzymes (Biofeed Plus, Novo Nordisk) added at the level of 1000 g/kg. All samples were taken to 65° C., held at temperature for 10 minutes with the temperature then increased to 85° C. and held for a further 5 minutes to de-activate the enzyme. Two of the samples were soaked (samples with SK prefix) for 24 hours in water prior to the microwave treatment. The samples were processed in 10 kg batches with 10 litres of water added to the bran prior to the microwave treatment. The microwave frequency used was 2.45 GHz. The results for free sugar release are presented in Table 2.

TABLE 2
Effects of enzyme supplementation and microwave treatment on the
carbohydrate composition of rice bran in a large scale operation

| | | | | | | | Total |
|---|---|---|---|---|---|---|---|
| | | | | | | | Sugars |
| | | | | | | | Released |
| SAMPLE | RIB | ARA | XYL | MAN | GAL | GLU | (g/kg) |
| Control 1 | 0 | 0 | 0 | 2.72 | 0 | 5.44 | 8.15 |
| Control 2 | 0 | 0 | 0 | 1.80 | 0 | 4.44 | 6.23 |
| Mean | 0 | 0 | 0 | 2.26 | 0 | 4.92 | 7.19 |
| Rice Bran | 0.68 | 0.83 | 0.49 | 16.49 | 1.11 | 67.43 | 87.03 |
| SK-1 | | | | | | | |
| Rice Bran | 0.59 | 0.81 | 0.46 | 15.03 | 0.92 | 68.70 | 86.51 |
| SK-2 | | | | | | | |
| Mean | 0.64 | 0.82 | 0.48 | 15.76 | 1.01 | 68.07 | 86.77 |
| Rice Bran | 0.68 | 0.50 | 0.28 | 14.64 | 1.06 | 60.77 | 77.93 |
| MV-1 | | | | | | | |
| Rice Bran | 0.71 | 0.57 | 0.37 | 14.53 | 1.24 | 61.57 | 78.99 |
| MV-2 | | | | | | | |
| Mean | 0.70 | 0.54 | 0.33 | 14.58 | 1.15 | 61.17 | 78.46 |

Results: The treatment was more effective than in Experiment 1, despite the fact the batch size had doubled. A 24 hour soaking of the rice bran at room temperature led to a small increase in free sugar release.

### Experiment 3

Aim: Given the promising results obtained from the previous experiments it was decided to undertake a detailed analysis of the improvement in the nutritive value of rice bran for poultry using a combined enzyme and microwave treatment.

Method: Two lots of rice bran (250 kg each), one with enzyme (Biofeed Plus, Novo Nordisk) supplementation (1000 g/tonne), were subjected to microwave treatment. The rice bran was processed in 10 kg batches with 10 litres of water added prior to the microwave treatment. The microwave frequency used was 2.45 GHz. All samples were taken to 65° C. and maintained at this temperature for 20 minutes. The rice bran contained 13.7% protein and a total of 26.6% NSP which consisted of 21.1% arabinoxylans, 0.4% mannose, 1.3% galactose and 12.8% cellulose plus trace amounts of ribose. In excess of 97% of the NSP were insoluble. Free sugar analysis was undertaken on triplicate sub samples. The results are presented below in Table 3.

TABLE 3
Effects of enzyme supplementation and microwave treatment on the
carbohydrate composition of rice bran used for the feeding trial

| | | | | | | | Total |
|---|---|---|---|---|---|---|---|
| | | | | | | | Sugars |
| | | | | | | | Released |
| SAMPLE | RIB | ARA | XYL | MAN | GAL | GLU | (g/kg) |
| Control 1 | 0 | 0 | 0 | 2.72 | 0 | 5.44 | 8.15 |
| Control 2 | 0 | 0 | 0 | 1.80 | 0 | 4.44 | 6.23 |
| Mean | 0 | 0 | 0 | 2.26 | 0 | 4.92 | 7.19 |
| Enzyme + MV1 | t | t | t | 8.62 | 1.67 | 58.01 | 68.29 |
| Enzyme + MV2 | t | t | t | 8.67 | 1.70 | 58.51 | 68.88 |
| Enzyme + MV3 | t | t | t | 8.58 | 1.73 | 58.78 | 69.09 |
| Mean | | | | 8.62 | 1.70 | 58.43 | 68.75 |
| Control + MV1 | t | t | t | 7.62 | 1.52 | 55.90 | 65.04 |
| Control + MV2 | t | t | t | 7.74 | 1.45 | 55.96 | 65.15 |
| Control + MV3 | t | t | t | 7.78 | 1.51 | 56.59 | 65.88 |
| Mean | | | | 7.62 | 1.52 | 55.90 | 65.04 |

Results: The amounts of free sugars released from both samples (rice bran- with or without enzyme) by microwave treatment were very similar, which is consistent with previous findings.

### Experiment 4

Aim: To determine if free sugar release from canola meal samples could be enhanced by the addition of an enzyme and/or microwave treatment.

Method: Four lots of 2 kg canola meal samples with a commercial enzyme (‘Energex’ a Novo Nordisk beta-glucanase enzyme) added to two of the samples at a dosage rate of 800 g/tonne. Two litres of water were added to each sample prior to microwave treatment. All samples were taken to 68° C. and held at this temperature for five minutes, after which the temperature was increased to 85° C. and held there for a further five minutes. The microwave frequency used was 2.45 GHz. For the analysis of each treatment, duplicate samples were taken.

Results are shown in Tables 4, 5 and 6. From each lot duplicate samples were taken for testing. Testing conditions were control, control plus enzyme (E1, E2), control plus microwave (MV1, MV2) and control plus enzyme plus microwave. The same enzyme was used in the lots with or without microwave.

TABLE 4
Canola meal calculation of free sugars (g/kg)

| SAMPLE | RHA | FUC | RIB | ARA | XYL | MAN | GAL | GLU | NSP |
|---|---|---|---|---|---|---|---|---|---|
| Control 1 | 0 | 0 | 0.24 | 1.05 | 0.23 | 2.19 | 6.30 | 21.06 | 31.06 |
| Control 2 | 0 | 0 | 0.26 | 1.25 | 0.23 | 2.47 | 6.73 | 21.83 | 32.77 |
| Mean | 0 | 0 | 0.25 | 1.15 | 0.23 | 2.33 | 6.52 | 21.45 | 31.92 |
| Control + Enzyme | 0 | 0 | 0.30 | 1.60 | 0.68 | 4.16 | 7.83 | 28.50 | 43.06 |
| 1 (E1) | | | | | | | | | |
| Control + Enzyme | 0 | 0 | 0.27 | 1.27 | 0.56 | 4.18 | 8.20 | 30.04 | 44.52 |
| 2 (E2) | | | | | | | | | |
| Mean | 0 | 0 | 0.29 | 1.43 | 0.62 | 4.17 | 8.01 | 29.27 | 43.79 |
| Control + MV 1 | 0 | 0 | 0.30 | 1.35 | 0.25 | 2.66 | 7.83 | 33.60 | 45.99 |
| Control + MV 2 | 0 | 0 | 0.32 | 1.37 | 0.28 | 2.59 | 8.01 | 33.12 | 45.69 |
| Mean | 0 | 0 | 0.31 | 1.36 | 0.27 | 2.62 | 7.92 | 33.36 | 45.84 |
| Control + MV + E1 | 0 | 0 | 0.34 | 1.46 | 0.47 | 3.76 | 9.17 | 40.39 | 55.59 |
| Control + MV + E2 | 0 | 0 | 0.30 | 1.24 | 0.39 | 3.32 | 8.77 | 38.64 | 52.66 |
| Mean | 0 | 0 | 0.32 | 1.35 | 0.43 | 3.54 | 8.97 | 39.51 | 54.12 |

TABLE 5
Canola meal calculation of soluble NSP (g/kg)

| SAMPLE | RHA | FUC | RIB | ARA | XYL | MAN | GAL | GLU | NSP |
|---|---|---|---|---|---|---|---|---|---|
| Control 1 | 0.83 | 0.67 | 0.62 | 6.45 | 1.03 | 1.20 | 3.46 | 2.51 | 14.42 |
| Control 2 | 0.91 | 0.48 | 0.66 | 6.76 | 1.00 | 1.20 | 3.55 | 2.39 | 14.56 |
| Mean | 0.87 | 0.57 | 0.64 | 6.61 | 1.02 | 1.20 | 3.50 | 2.45 | 14.49 |
| Control + Enzyme 1 (E1) | 1.01 | 0.68 | 0.67 | 7.95 | 0.95 | 1.08 | 3.47 | 1.87 | 15.20 |
| Control + Enzyme 2 (E2) | 1.04 | 0.82 | 0.63 | 7.32 | 0.84 | 1.02 | 3.24 | 1.78 | 14.33 |
| Mean | 1.03 | 0.75 | 0.65 | 7.64 | 0.90 | 1.05 | 3.35 | 1.82 | 14.77 |
| Control + MV 1 | 1.42 | 0.68 | 0.68 | 7.45 | 1.43 | 2.65 | 3.45 | 2.79 | 17.04 |
| Control + MV 2 | 1.53 | 1.40 | 0.75 | 7.20 | 1.22 | 2.75 | 3.62 | 3.01 | 17.89 |
| Mean | 1.48 | 1.04 | 0.71 | 7.32 | 1.32 | 2.70 | 3.54 | 2.90 | 17.47 |
| Control + MV + E1 | 2.36 | 0.83 | 0.86 | 7.79 | 1.06 | 2.60 | 3.29 | 2.44 | 17.65 |
| Control + MV + E2 | 1.40 | 1.32 | 0.81 | 7.33 | 1.03 | 2.77 | 3.30 | 2.44 | 16.90 |
| Mean | 1.88 | 1.08 | 0.83 | 7.56 | 1.04 | 2.69 | 3.30 | 2.44 | 17.28 |

TABLE 6
Canola meal calculation of insoluble NSP (g/kg)

| SAMPLE | RHA | FUC | RIB | ARA | XYL | MAN | GAL | GLU | NSP |
|---|---|---|---|---|---|---|---|---|---|
| Control 1 | 3.49 | 2.03 | 0.31 | 48.04 | 19.72 | 5.35 | 17.96 | 36.80 | 118.94 |
| Control 2 | 5.11 | 3.30 | 0.66 | 45.27 | 19.88 | 4.31 | 17.36 | 60.15 | 139.04 |
| Mean | 4.30 | 2.67 | 0.48 | 46.66 | 19.80 | 4.83 | 17.66 | 48.48 | 128.99 |
| Control + Enzyme | 4.43 | 2.53 | 0.26 | 43.64 | 18.34 | 4.08 | 16.36 | 65.67 | 138.48 |
| 1 (E1) | | | | | | | | | |
| Control + Enzyme | 4.38 | 2.43 | 0.26 | 40.56 | 16.95 | 3.83 | 15.79 | 33.41 | 104.63 |
| 2 (E2) | | | | | | | | | |
| Mean | 4.40 | 2.48 | 0.26 | 42.10 | 17.64 | 3.96 | 16.08 | 49.54 | 121.55 |
| Control + MV 1 | 5.82 | 4.98 | 0.51 | 43.1 | 20.68 | 3.87 | 17.11 | 82.58 | 159.39 |
| Control + MV 2 | 5.19 | 4.33 | 0.61 | 54.92 | 22.59 | 4.83 | 17.35 | 68.92 | 159.21 |
| Mean | 5.50 | 4.66 | 0.56 | 49.01 | 21.64 | 4.35 | 17.23 | 75.75 | 159.30 |
| Control + MV + E1 | 3.19 | 2.57 | 0.21 | 43.06 | 18.10 | 4.24 | 18.05 | 83.45 | 154.30 |
| Control + MV + E2 | 2.63 | 1.83 | 0.24 | 41.99 | 18.20 | 4.12 | 17.24 | 80.91 | 149.19 |
| Mean | 2.91 | 2.20 | 0.22 | 42.53 | 18.15 | 4.18 | 17.64 | 82.18 | 151.74 |

Results: Table 4 suggests that enzymatic treatment released more glucose relative to the control samples. Slightly increased glucose levels were obtained with microwave treatment. The highest levels of glucose were obtained by the microwave treatment of a canola meal/enzyme mix. Similar results, but at lower relative levels were obtained for galactose.

Table 5 results indicated that the microwave treatment can have some effect without the presence of enzymes.

Table 6 results indicated that microwave treatment and the microwave treatment of a canola meal/enzyme mix produced the highest levels of glucose release.

Treatment using microwave energy and enzymes released more sugars from cellulosic material thus allowing greater ethanol production from the treated material. If there are more free sugars available, then more ethanol production is possible as there is more starting sugars available for fermentation.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A process for converting cellulosic material to ethanol comprising:
(a) adding an enzyme capable of releasing sugars to cellulosic material to form a cellulosic material and enzyme mixture;
(b) treating the mixture with microwave energy to enhance enzymatic digestion of the cellulosic material by the enzyme to release sugars; and
(c) carrying out a fermentation reaction on the treated mixture to form ethanol.

2. The process according to claim 1 wherein the cellulosic material is a cellulosic biomass feedstock selected from agricultural waste, plant waste from industrial processes, an energy crop, components thereof, or mixtures thereof.

3. The process according to claim 2 wherein the agricultural waste is corn stover, cereal straw or sugarcane bagasse, the plant waste from industrial processes is saw dust or paper pulp, and the energy crop is switch grass.

4. The process according to claim 3 wherein the cellulosic material is corn stover.

5. The process according to according to claim 1 wherein the enzyme is selected from the group consisting of amylase, alpha amylase, glucoamylase, phytase, phosphatase, carbohydrate hydrolyzing enzyme, xylanase, cellulose, hemi-cellulase and mixtures or combinations thereof.

6. The process according to claim 5 wherein the enzyme is an alpha amylase.

7. The process according to claim 1 wherein the enzyme is added at a range from 500 g to 5000 g per tonne.

8. The process according to claim 7 wherein the amount of enzyme is added at 1000 g to 2000 g per tonne.

9. The process according to claim 1 wherein the microwave energy has a frequency of 2.45 GHz or in the 900 MHz frequency range.

10. The process according to claim 1 wherein the treating with microwave energy is carried out such that the temperature of the mixture is effectively controlled.

11. The process according to claim 10 wherein the temperature is from 65° C. to 78° C.

12. The process according to claim 11 wherein the temperature is about 70° C.

13. The process according to claim 1 wherein the treating with microwave energy is carried out in a continuous or batch manner.

14. The process according to claim 13 wherein the treating with microwave energy is carried out for up to about 10 minutes per kg cellulosic material.

15. The process according to claim 1 wherein the fermentation reaction is carried out by microbial fermentation.

16. The process according to claim 15 wherein the microbial fermentation utilizes yeast.

17. The process according to claim 1 carried out in wet milling ethanol production.

18. The process according to claim 1 carried out in dry milling ethanol production.

19. Ethanol produced from cellulosic material by a process comprising:
(a) adding an enzyme capable of releasing sugars to cellulosic material to form a cellulosic material and enzyme mixture;
(b) treating the mixture with microwave energy to enhance enzymatic digestion of the cellulosic material by the enzyme to release sugars; and
(c) carrying out a fermentation reaction on the treated mixture to form ethanol.
